SEQUENCE LISTING (8 sequences)

SEQ ID NO: 1 - DNA, 60 bp, Artificial Sequence
  Synthetic sequence
cgtacgtattgctagcataagtcagctatgacttcgatcagcatatctacggcatagcat

SEQ ID NO: 2 - DNA, 60 bp, Artificial Sequence
  Synthetic sequence
cgtacgtattgctagcataagtcagctatgacttcgattagcatatctacggcatagcat

SEQ ID NO: 3 - DNA, 60 bp, Artificial Sequence
  Synthetic sequence
cgtacgtattgctagcataaggcagctatgacttcgattagcatatctacggcatagcat

SEQ ID NO: 4 - DNA, 60 bp, Artificial Sequence
  Synthetic sequence
cgtacgtattgctagcataagtcagctatgacttcgatcagcatgcatagcatatggcca

SEQ ID NO: 5 - DNA, 60 bp, Artificial Sequence; identical to SEQ ID NO: 2
  Synthetic sequence

SEQ ID NO: 6 - DNA, 60 bp, Artificial Sequence
  Synthetic sequence
cgtacgtattgctagcataaggcagctatgacttcgattagcatgcatagcatatggcca

SEQ ID NO: 7 - DNA, 148 bp, Artificial Sequence
  Synthetic sequence
aractaggattttggtcacttctarratggracatttaargrragctgrcarartattarttttgcrtgaaggtrgcrctatttttatgggrcrttttcrgrrctccarartctacagccrgrctttagctcaarrctcatgggatgt

SEQ ID NO: 8 - protein, 50 aa, Artificial Sequence
  Synthetic sequence
KTRILVTSKHEHLKKADKILILHEGSSYFYGTFSELQNLQPDFSSKLHGC

(19) United States
(12) Patent Application Publication
(10) Pub. No.: US 2019/0295690 A1
(43) Pub. Date: Sep. 26, 2019

(54) VARIANT DETECTION OF SEQUENCING ASSAYS

(71) Applicant: Good Start Genetics, Inc., Cambridge, MA (US)

(72) Inventors: Athurva Gore, Cambridge, MA (US); Eric Tsung, Cambridge, MA (US); Gregory Porreca, Cambridge, MA (US)

(21) Appl. No.: 16/074,370

(22) PCT Filed: Feb. 3, 2017
(86) PCT No.: PCT/US2017/016498

§ 371 Date: Jul. 31, 2018

Related U.S. Application Data
(60) Provisional application No. 62/292,096, filed on Feb. 5, 2016.

(51) Int. Cl. G16B 30/10; G16B 30/20; G16B 40/00; G16B 45/00

(52) U.S. Cl. CPC G16B 30/10; G16B 45/00; G16B 40/00; G16B 30/20

(57) ABSTRACT

Methods for improving variant protection are provided. The methods include the steps of generating an in silico genome map that includes a primary path, and a secondary path comprising variants that were missed or incorrectly called in a sequence assembly simulation, aligning the secondary path to the primary path, convoluting the second path to the primary path, and identifying the variants based upon reads that align with the secondary path but not the primary path. All of method steps can be carried out using a computer. Systems incorporated these methods are also included.

### RELATED APPLICATIONS

The present application is a 35 U.S.C. § 371 national phase entry of PCT/US2017/016498, with international filing date Feb. 3, 2017, which claims the benefit of and priority to U.S. provisional patent application Ser. No. 62/292,096, filed Feb. 5, 2016, the content of each of which is incorporated by reference herein in its entirety.

### SEQUENCE LISTING

This application contains a sequence listing which has been submitted in ASCII format via EFS-Web and is hereby incorporated by reference in its entirety. The ASCII-formatted sequence listing, created on Apr. 11, 2019, is named GSGE-035-Sequence-Listing, and is 4,096 bytes in size.

### FIELD OF THE INVENTION

The invention generally relates to methods and systems for improving detection of variants of sequencing assays.

### BACKGROUND

Genetic testing provides individuals with valuable information. For example, carrier screening can determine whether one or both members of a couple are carriers of a recessive genetic disorder. With this information, the couple can determine whether they are at risk for having children with the genetic disorder. Pre-implantation genetic screening also provides couples with valuable information about the viability of their embryos when undergoing in vitro fertilization (IVF) treatments. The information provided can help bring the couple closer to achieving pregnancy by identifying the healthiest embryos for transfer. As new technologies such as next-generation sequencing (NGS) emerge, those technologies promise to provide more rapid and affordable genetic tests.

Next generation sequencing has revolutionized clinical variant detection. Many rare or complex pathogenic variants can now be interrogated with a single assay. However, the sheer diversity of pathogenic variants poses a major challenge to evaluating the accuracy of genotype calling in high-througput clinical sequencing assays, such as NGS. Some rare and/or complex variations by their very nature are problematic to detect. Many clinical NGS data processing pipelines use a variant discovery approach, such as the approach disclosed in FIG. 1. In this approach, raw sequencing reads from each sample are first mapped to a reference sequence. Locations where the consensus sequence of a sample differs from the reference sequence are then identified. Finally, the variants are annotated to determine pathogenicity, including functional annotation and comparison to reference databases. However, because many important variants are extremely rare, samples bearing such variants are often not available. This begs the question of how one can be sure that a clinical assay will detect these rare variants?

While in silico simulation-based approaches have previously been used as a proxy for having positive samples, these approaches often introduce alleles into simulated reads derived from a reference genome, and may not represent the real world characteristics and error modes of a sequencing run. Therefore, methods for accurately evaluating whether an analysis pipeline is capable of detecting complex and rare variations and for improving the detection sensitivity of a variation analysis pipeline are desirable.

### SUMMARY

The invention provides methods and systems for improving the calling of complex variants using a graph-based alignment approach. Using this approach, variants of interest (known to exist in the population, which could have low detection sensitivity with traditional analysis approaches) are added to a genomic graph as separate nodes. In this way, the calling of many large insertions, deletions, and complex INDELs not easily callable by traditional alignment-based analysis pipelines or assembly-based approaches can be improved.

In certain embodiments, the invention provides a method for improving variant detection of a genetic test. The method involves sequencing nucleic acid from a sample to produce a plurality of sequence reads, introducing one or more simulated variants into at least one of the plurality of sequence reads, analyzing the reads to determine whether the simulated variants were detected, adding any variants that were not detected or were incorrectly called to a graph genome as a secondary path, or node, and analyzing the reads again to determine whether the test is able to identify the variants. In certain aspects, any or all of the steps are carried out using a computer comprising a non-transitory memory.

In other embodiments, the invention provides methods for improving variant detection of a genetic test by adding one or more variants of interest to a graph genome as a set of secondary paths, representing one or more alternate genomic features, or node, and analyzing a plurality of sequence reads generated by sequencing nucleic acid from a sample to determine whether the test is able to identify the variants.

Analysis of sequence reads to determine whether the test is able to identify the simulated variants that were not detected or were improperly called can include the step of assembling the plurality of sequence reads to produce a contig. The contig can be aligned to either the primary path or the secondary path of the graph genome. In one aspect, the plurality of sequence reads can be aligned to the contig. The aligned sequence reads can then be convoluted to the primary path of the graph genome.

In some aspects, analysis of sequence reads to determine whether the test is able to identify the simulated variants that were not detected or were improperly called can include the step of assembling the plurality of sequence reads and aligning the one or more of the plurality of sequence reads to either the primary path or the secondary path of the graph genome. The aligned sequence reads can then be convoluted to the primary path of the graph genome and these reads can also be combined with reads aligned via another methodology, including the methodology Genotyping by Assembly Templated Alignment (GATA) to generate the final genotype call(s).

In another aspect of the invention, the methods are carried out using a computer comprising a non-transitory memory, such that the computer is operable to detect a variant and report the variant in relation to the genome.

### BRIEF DESCRIPTION OF THE DRAWINGS

FIG. 1 illustrates a basic variant discovery approach using an NGS data processing pipeline

FIG. 2 illustrates the alignment of reads to a graph genome.

FIG. 3 is a diagram of a method for validating a variant analysis pipeline

FIG. 4 illustrates the simulation of variants into real reads.

FIG. 5 shows a computer system in accordance with embodiments of the invention.

FIG. 6 illustrates the detection of the CFTR:c.1817_1900de184 variant.

### DETAILED DESCRIPTION

For a genetic analysis method such as an NGS platform to be used for clinical genetic testing, it must satisfy at least three requirements. First, analytical accuracy must be both high and well characterized within the clinically relevant genes or regions. Second, sequencing technologies should yield data sufficient to cover the vast majority of targeted bases at a depth sufficient to make high-quality genotype calls while computational technologies should have power commensurate with the yield of sequencing technologies. Finally, workflow should be robust and reproducible, which can often be achieved through automation.

Systems and methods of the invention may be used to evaluate the validity of (e.g., validate) an analytical method, system, or combination thereof used in genetic testing and to improve the sensitivity of such method, system or combination thereof. Methods of the invention include testing the detectability of a mutation. For example, where a mutation is known or suspected, whether it is abundant, rare, very rare, or of unknown prevalence, the invention provides the ability to evaluate whether that mutation will be detected by known systems and methods, as well as the ability to improve the detection sensitivity of a system or method.

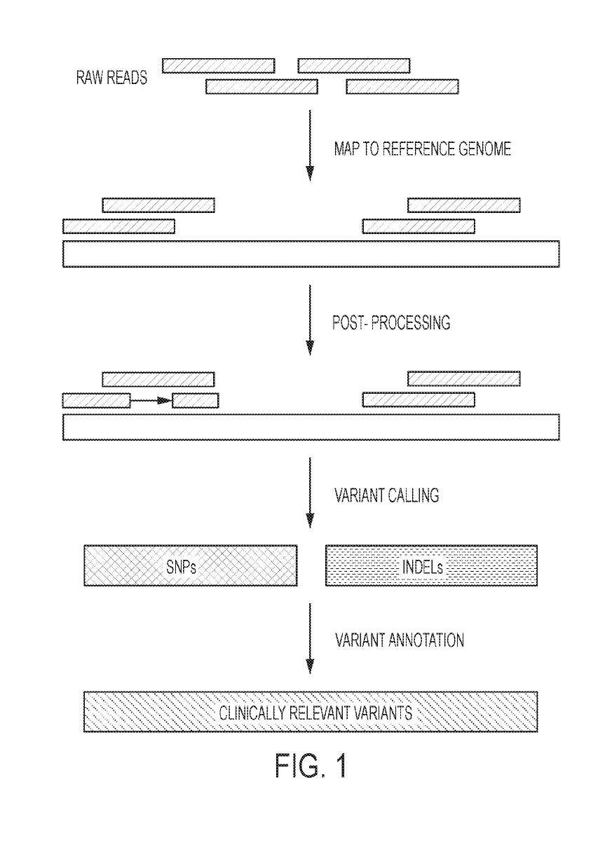

Many clinical data processing pipelines using an NGS platform utilize a variant discovery approach. As shown in FIG. 1, this approach generally involves obtaining raw sequencing reads from each sample. The raw reads are then mapped to a reference sequence. Variants are identified at locations where the consensus sequence of a sample differs from the reference sequence. The variants are then annotated to determine pathogenicity, including functional annotation and comparison to various reference databases.

However, the use of this type of linear representation (e.g., reference sequence) has its limitations. While the sequences of chromosomes between individuals are similar, there are many differences. Some of these variations can be small, such as single nucleotide polymorphism (SNPs), small insertions and deletions (indels), and copy number variations. However, some variations are much larger, spanning larger sequences, such as chromosomal rearrangements (deletions, duplications, inversions and translocations). Representation of a diploid (two sets of chromosomes) genome using a single linear sequence leaves out some of this variation, or additional ways of reporting the variation become necessary. Furthermore, sequencing a new genome in which sequences may not be represented in the reference may result in parts of the sample sequence being missing.

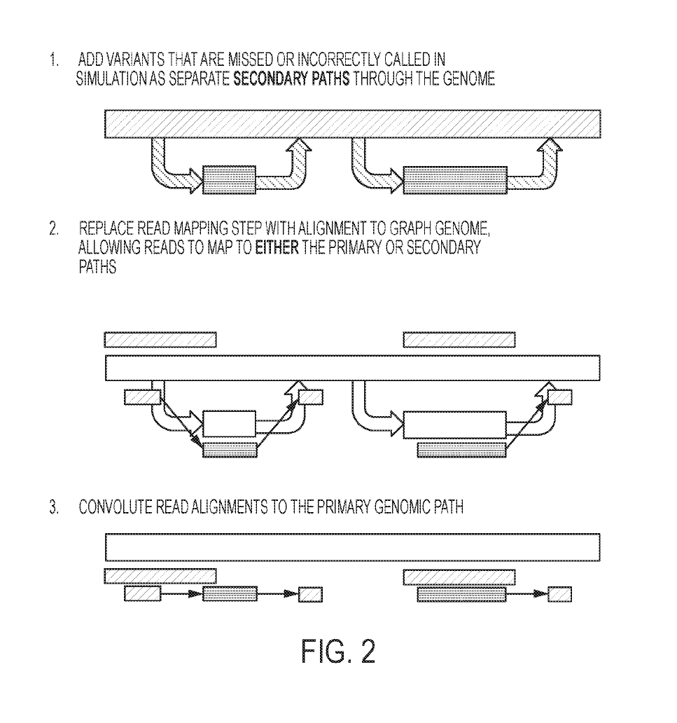

By introducing a graph-based alignment method, the calling of complex variants can be improved compared to linear based variant calling, due to the ability to provide for alternative paths for regions where the chromosomes differ. FIG. 2 illustrates the alignment of reads to a graph genome according to the invention. As shown, variants that are missed or incorrectly called in a simulation can be added as separate secondary paths (or nodes) through the genome. Next, the typical read mapping step is replaced with alignment to a graph genome. Such alignment allows for reads to map to either the primary or secondary path. It is to be understood that many alternate, or secondary, paths can be provided in accordance with the invention, each secondary path representing a genome with a different alternate variant. Read alignments are then convoluted to the primary genomic path.

In certain aspects, the invention provides a method of evaluating an analysis pipeline for its ability to identify pathogenic variants by introducing simulated variants. The method involves obtaining a plurality of sequence reads, introducing simulated variants into the plurality of sequence reads, and analyzing the sequence reads to determine if the analysis pipeline identifies the simulated variants. The variants can be introduced to the plurality of sequence reads at a desired allele fraction. The simulated mutation can be introduced by manipulating a data field in the sequence read such as, for example, a base sequence field or quality data field. The sequences can be manipulated by a computer program. For example, a program can be written using Java, Groovy, Python, Perl, or other languages, or a combination thereof, that can automatically insert simulated mutations into sequence reads. Computer-based methods can be used to automatically introduce a number of different simulated mutations into different ones of the plurality of sequence reads. The sequence reads are then ran through the variant calling pipeline to ensure that the simulated variant is called.

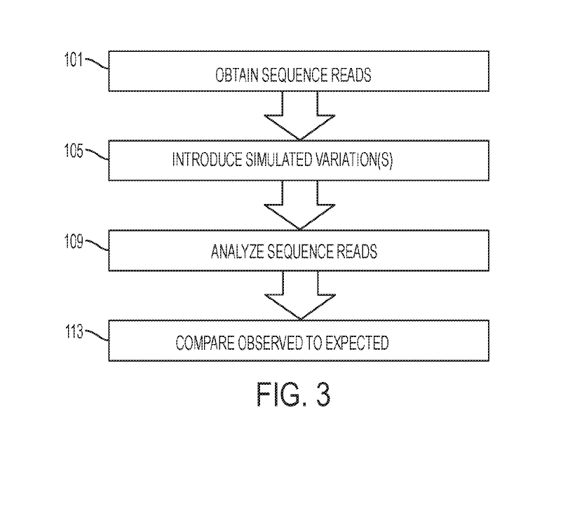

FIG. 3 is a diagram of a method of evaluating an analysis pipeline. In general, methods of the invention include obtaining sequence reads (101) and introducing a simulated variation into at least one of the reads (105). Sequence reads may be obtained by any method including loading into computer memory files from prior work or receiving computer files from an outside source. In certain embodiments, the sequence reads are obtained 101 by isolating nucleic acid from a sample and sequencing the nucleic acid.

Nucleic acid template molecules (e.g., DNA or RNA) can be isolated from a sample containing other components, such as proteins, lipids and non-template nucleic acids. Nucleic acid can be obtained directly from a patient or from a sample such as blood, urine, cerebrospinal fluid, seminal fluid, saliva, sputum, stool and tissue. Any tissue or body fluid specimen may be used as a source for nucleic acid. Nucleic acid can also be isolated from cultured cells, such as a primary cell culture or a cell line. Generally, nucleic acid can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, N.Y. 2,028 pages (2012); or as described in U.S. Pat. Nos. 7,957,913; 7,776,616; 5,234,809; U.S. Pub. 2010/0285578; and U.S. Pub. 2002/0190663.

Nucleic acid obtained from biological samples may be fragmented to produce suitable fragments for analysis. Template nucleic acids may be fragmented or sheared to desired length, using a variety of mechanical, chemical and/or enzymatic methods. Nucleic acid may be sheared by sonication, brief exposure to a DNase/RNase, hydro shear instrument, one or more restriction enzymes, transposase or nicking enzyme, exposure to heat plus magnesium, or by shearing.

A biological sample may be lysed, homogenized, or fractionated in the presence of a detergent or surfactant as needed. Suitable detergents may include an ionic detergent (e.g., sodium dodecyl sulfate or N-lauroylsarcosine) or a nonionic detergent (such as the polysorbate 80 sold under the trademark TWEEN by Uniqema Americas (Paterson, N.J.) or C₁₄H₂₂O (C₂H₄)ₙ, known as TRITON X-100). Once a nucleic acid is extracted or isolated from the sample it may be amplified.

Amplification refers to production of additional copies of a nucleic acid sequence and is generally carried out using polymerase chain reaction (PCR) or other technologies known in the art. The amplification reaction may be any amplification reaction known in the art that amplifies nucleic acid molecules such as PCR. Other amplification reactions include nested PCR, PCR-single strand conformation polymorphism, ligase chain reaction, strand displacement amplification and restriction fragments length polymorphism, transcription based amplification system, rolling circle amplification, and hyper-branched rolling circle amplification, quantitative PCR, quantitative fluorescent PCR (QF-PCR), multiplex fluorescent PCR (MF-PCR), real time PCR (RTPCR), restriction fragment length polymorphism PCR (PCR-RFLP), in situ rolling circle amplification (RCA), bridge PCR, picotiter PCR, emulsion PCR, transcription amplification, self-sustained sequence replication, consensus sequence primed PCR, arbitrarily primed PCR, degenerate oligonucleotide-primed PCR, and nucleic acid based sequence amplification (NABSA). Amplification methods that can be used include those described in U.S. Pat. Nos. 5,242,794; 5,494,810; 4,988,617; and 6,582,938. In certain embodiments, the amplification reaction is PCR as described, for example, U.S. Pat. Nos. 4,683,195; and 4,683,202, hereby incorporated by reference. Primers for PCR, sequencing, and other methods can be prepared by cloning, direct chemical synthesis, and other methods known in the art. Primers can also be obtained from commercial sources such as Eurofins MWG Operon (Huntsville, Ala.) or Life Technologies (Carlsbad, Calif.).

With these methods, a single copy of a specific target nucleic acid may be amplified to a level that can be sequenced. Further, the amplified segments created by an amplification process such as PCR may be, themselves, efficient templates for subsequent PCR amplifications.

Amplification or sequencing adapters or barcodes, or a combination thereof, may be attached to the fragmented nucleic acid. Such molecules may be commercially obtained, such as from Integrated DNA Technologies (Coralville, Iowa). In certain embodiments, such sequences are attached to the template nucleic acid molecule with an enzyme such as a ligase. Suitable ligases include T4 DNA ligase and T4 RNA ligase, available commercially from New England Biolabs (Ipswich, Mass.). The ligation may be blunt ended or via use of complementary overhanging ends. In certain embodiments, following fragmentation, the ends of the fragments may be repaired, trimmed (e.g. using an exonuclease), or filled (e.g., using a polymerase and dNTPs) to form blunt ends. In some embodiments, end repair is performed to generate blunt end 5′ phosphorylated nucleic acid ends using commercial kits, such as those available from Epicentre Biotechnologies (Madison, Wis.). Upon generating blunt ends, the ends may be treated with a polymerase and dATP to form a template independent addition to the 3′-end and the 5′-end of the fragments, thus producing a single A overhanging. This single A can guide ligation of fragments with a single T overhanging from the 5′-end in a method referred to as T-A cloning. Alternatively, because the possible combination of overhangs left by the restriction enzymes are known after a restriction digestion, the ends may be left as-is, i.e., ragged ends. In certain embodiments double stranded oligonucleotides with complementary overhanging ends are used.

In certain embodiments, one or more bar code is attached to each, any, or all of the fragments. A bar code sequence generally includes certain features that make the sequence useful in sequencing reactions. The bar code sequences are designed such that each sequence is correlated to a particular portion of nucleic acid, allowing sequence reads to be correlated back to the portion from which they came. Methods of designing sets of bar code sequences is shown for example in U.S. Pat. No. 6,235,475, the contents of which are incorporated by reference herein in their entirety. In certain embodiments, the bar code sequences range from about 5 nucleotides to about 15 nucleotides. In a particular embodiment, the bar code sequences range from about 4 nucleotides to about 7 nucleotides. In certain embodiments, the bar code sequences are attached to the template nucleic acid molecule, e.g., with an enzyme. The enzyme may be a ligase or a polymerase, as discussed above. Attaching bar code sequences to nucleic acid templates is shown in U.S. Pub. 2008/0081330 and U.S. Pub. 2011/0301042, the content of each of which is incorporated by reference herein in its entirety. Methods for designing sets of bar code sequences and other methods for attaching bar code sequences are shown in U.S. Pat. Nos. 6,138,077; 6,352,828; 5,636,400; 6,172,214; 6,235,475; 7,393,665; 7,544,473; 5,846,719; 5,695,934; 5,604,097; 6,150,516; RE39,793; 7,537,897; 6172,218; and 5,863,722, the content of each of which is incorporated by reference herein in its entirety. After any processing steps (e.g., obtaining, isolating, fragmenting, amplification, or barcoding), nucleic acid can be sequenced.

Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Separated molecules may be sequenced by sequential or single extension reactions using polymerases or ligases as well as by single or sequential differential hybridizations with libraries of probes.

A sequencing technique that can be used includes, for example, use of sequencing-by-synthesis systems sold under the trademarks GS JUNIOR, GS FLX+ and 454 SEQUENCING by 454 Life Sciences, a Roche company (Branford, Conn.), and described by Margulies, M. et al., Genome sequencing in micro-fabricated high-density picotiter reactors, Nature, 437:376-380 (2005); U.S. Pat. Nos. 5,583,024; 5,674,713; and 5,700,673, the contents of which are incorporated by reference herein in their entirety. 454 sequencing involves two steps. In the first step of those systems, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

Another example of a DNA sequencing technique that can be used is SOLiD technology by Applied Biosystems from Life Technologies Corporation (Carlsbad, Calif.). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is removed and the process is then repeated.

Another example of a DNA sequencing technique that can be used is ion semiconductor sequencing using, for example, a system sold under the trademark ION TORRENT by Ion Torrent by Life Technologies (South San Francisco, Calif.). Ion semiconductor sequencing is described, for example, in Rothberg, et al., An integrated semiconductor device enabling non-optical genome sequencing, Nature 475:348-352 (2011); U.S. Pub. 2010/0304982; U.S. Pub. 2010/0301398; U.S. Pub. 2010/0300895; U.S. Pub. 2010/0300559; and U.S. Pub. 2009/0026082, the contents of each of which are incorporated by reference in their entirety.

Another example of a sequencing technology that can be used is Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated. Sequencing according to this technology is described in U.S. Pat. Nos. 7,960,120; 7,835,871; 7,232,656; 7,598,035; 6,911,345; 6,833,246; 6,828,100; 6,306,597; 6,210,891; U.S. Pub. 2011/0009278; U.S. Pub. 2007/0114362; U.S. Pub. 2006/0292611; and U.S. Pub. 2006/0024681, each of which are incorporated by reference in their entirety.

Another example of a sequencing technology that can be used includes the single molecule, real-time (SMRT) technology of Pacific Biosciences (Menlo Park, Calif.). In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a sequencing technique that can be used is nanopore sequencing (Soni, G. V., and Meller, A., Clin Chem 53: 1996-2001 (2007)). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

Another example of a sequencing technique that can be used involves using a chemical-sensitive field effect transistor (chemFET) array to sequence DNA (for example, as described in U.S. Pub. 2009/0026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Another example of a sequencing technique that can be used involves using an electron microscope as described, for example, by Moudrianakis, E. N. and Beer M., in Base sequence determination in nucleic acids with the electron microscope, III. Chemistry and microscopy of guanine-labeled DNA, PNAS 53:564-71 (1965). In one example of the technique, individual DNA molecules are labeled using metallic labels that are distinguishable using an electron microscope. These molecules are then stretched on a flat surface and imaged using an electron microscope to measure sequences.

Sequencing generates a plurality of reads. Reads generally include sequences of nucleotide data less than about 150 bases in length, or less than about 90 bases in length. In certain embodiments, reads are between about 80 and about 90 bases, e.g., about 85 bases in length. In some embodiments, these are very short reads, i.e., less than about 50 or about 30 bases in length. With continued reference to FIG. 1, after obtaining 101 sequence reads, a simulated variant(s) is introduced into one or more of the reads. A simulated variant can include a change (an “edit”) to data within a read file. A read file may be edited to correspond to known genotype—the “expected” genotype. Any expected genotype can be used and any simulation can be introduced into the sequence reads including, for example, single variations or combinations of variations.

Then the sequence reads are analyzed to determine a genotype (the “observed” genotype). A set of sequence reads can be analyzed by any suitable method known in the art. For example, in some embodiments, sequence reads are analyzed by hardware or software provided as part of a sequence instrument. In some embodiments, individual sequence reads are reviewed by sight (e.g., on a computer monitor). A computer program may be written that pulls an observed genotype from individual reads. In certain embodiments, analyzing the reads includes assembling the sequence reads and then genotyping the assembled reads.

Sequence assembly can be done by methods known in the art including reference-based assemblies, de novo assemblies, assembly by alignment, or combination methods. Assembly can include methods described in U.S. Pat. No. 8,209,130 titled Sequence Assembly by Porecca and Kennedy, the contents of each of which are hereby incorporated by reference in their entirety for all purposes. In some embodiments, sequence assembly uses the low coverage sequence assembly software (LOCAS) tool described by Klein, et al., in LOCAS-A low coverage sequence assembly tool for re-sequencing projects, PLoS One 6(8) article 23455 (2011), the contents of which are hereby incorporated by reference in their entirety. Sequence assembly is described in U.S. Pat. Nos. 8,165,821; 7,809,509; 6,223,128; U.S. Pub. 2011/0257889; and U.S. Pub. 2009/0318310, the contents of each of which are hereby incorporated by reference in their entirety.

Making reference to FIG. 1, analyzing the sequence reads (e.g., after assembly) (109) includes obtaining an observed genotype. By comparing the observed genotype to the expected genotype (113), the validity of the analytical pipeline can be assessed.

One insight of the invention is that, by introducing simulated variations into the sequence reads, genetic context is preserved. Genetic context can actually influence the success or outcome of an analysis. For example, where a sample of genetic material contains a single nucleotide polymorphism SNP relative to a reference, with no other variants in proximity to the SNP, some analytical pipelines will detect that SNP in the sample material. However, where that SNP is very close to (e.g., within 25 or fewer, such as within about 5 base-pairs of) an insertion or deletion (indel), those same analytical pipelines may fail to detect the SNP in the sample material. Thus, it is possible that the genetic context of the SNP interferes with the ability of a genetic test to detect the mutation.

Methods of the invention include introducing a simulated variation (e.g., the SNP and proximal indel just discussed), or a list of variants that can be introduced at a desired allele fraction, into the sequence reads. This way, the genetic context is preserved. For example, if a read assembly algorithm fails to detect the inserted SNP when it is proximal to an indel, by including the genetic context provided by the actual reads from the sequencing instrument run, the failure of the algorithm will be revealed.

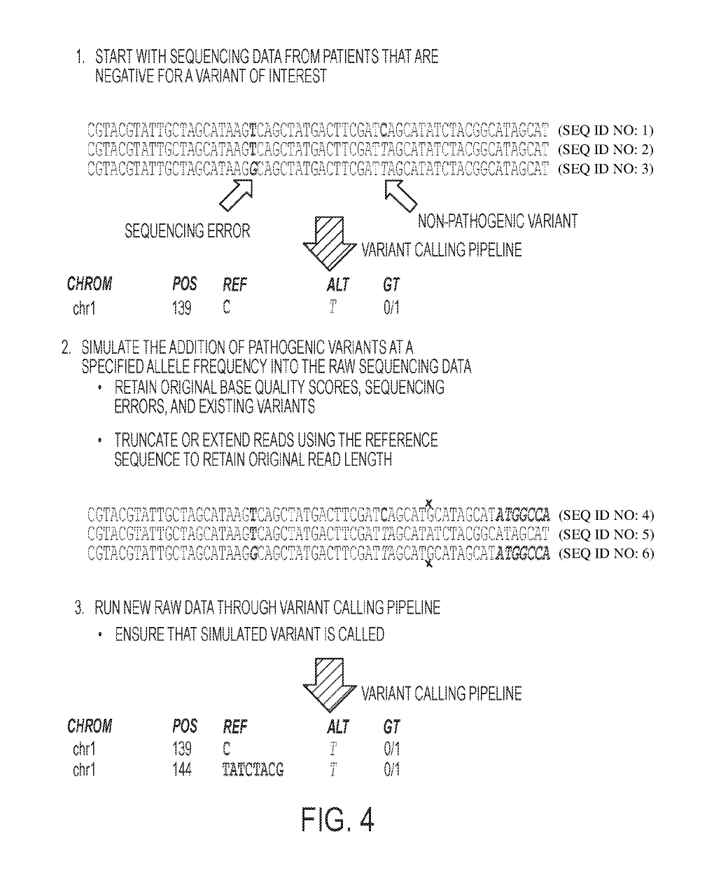

FIG. 4 depicts a hypothetical example of introducing a simulated variant into the sequence reads. In this hypothetical example, samples from patients that are negative for a variant of interest are sequenced to provide sequencing data (step 1.). The addition of pathogenic variants at a specified allele frequency is simulated into the raw sequencing data (step 2.). The original base quality score is to be retained, as are the sequencing errors and existing variants. As shown, reads can be truncated or extended using the reference sequence to retain the original read length. Finally the new raw data is run through a full analysis pipeline to determine whether the simulated variant is called (step 3). By running the files through a full analysis pipeline, end-to-end simulation in the context of actual sequencing/capture error modes is allowed. If it is found that the simulated variant is missed or incorrectly called, it is revealed that the variant analysis pipeline is not sensitive to the variant of interest.

It is theorized that the sensitivity of an NGS analysis pipeline relates to the nature of the mutations present, the sequencing coverage, and sequencing errors. For example, some mutations by their nature are particularly difficult for some NGS platforms to pick up. A very long indel, an indel at or near an end of a read, or a substitution close to a long indel can produce reads that NGS platforms do not assemble correctly. Some sequencing methodologies are prone to sequencing errors when sequencing polynucleotide repeats.

Systems and methods of the invention may be used to improve upon the sensitivity of variant analysis pipelines, such as NGS-based assays. While sequence assembly can be done by any of the methods known in the art, including reference-based assemblies, de novo assemblies and assembly by alignment, preferred systems and methods of the invention use a graph-based alignment method (‘GATAgraph’ method) to call variants.

FIG. 2 illustrates the use of a graph-based alignment method to call variants. As shown in Step 1 of FIG. 2, any simulated variants that are missed or incorrectly called can be added as one or more separate secondary paths (or nodes) through the graph genome. In this method, rather than mapping the reads solely to a linear reference genome, reads can be mapped to either the primary path through graph genome or an added secondary path, or node.

In one embodiment, the reads can first be assembled into contigs. The contigs can then be aligned to the primary or secondary path through the graph genome. The raw reads can then be aligned to the contigs. The raw read alignments are then mapped from the contig space to the genome. This method is similar to the Genotyping by Assembly-Templated Alignment (GATA) method disclosed in U.S. Patent Publication No. 2014/0129201 and Umbarger et al., (2013) Next-generation carrier screening, Genetics in Medicine, 16(2), 132-140., both of which are incorporated herein in their entirety, yet differs in that the GATAgraph method includes the use of a graph genome allowing for separate nodes to be added.

Reads may be assembled into contigs by any method known in the art. Algorithms for the de novo assembly of a plurality of sequence reads are known in the art. One algorithm for assembling sequence reads is known as overlap consensus assembly. Assembly with overlap graphs is described, for example, in U.S. Pat. No. 6,714,874. In some embodiments, de novo assembly proceeds according to so-called greedy algorithms, as described in U.S. Pub. 2011/0257889, incorporated by reference in its entirety. In other embodiments, assembly proceeds by either exhaustive or heuristic pairwise alignment. Exhaustive pairwise alignment, sometimes called a “brute force” approach, calculates an alignment score for every possible alignment between every possible pair of sequences among a set. Assembly by heuristic multiple sequence alignment ignores certain mathematically unlikely combinations and can be computationally faster. One heuristic method of assembly by multiple sequence alignment is the so-called “divide-and-conquer” heuristic, which is described, for example, in U.S. Pub. 2003/0224384. Another heuristic method of assembly by multiple sequence alignment is progressive alignment, as implemented by the program ClustalW (see, e.g., Thompson, et al., Nucl. Acids. Res., 22:4673-80 (1994)).

In some embodiments, assembly into contigs involves making a de Bruijn graph. De Bruijn graphs reduce the computation effort by breaking reads into smaller sequences of DNA, called k-mers, where the parameter k denotes the length in bases of these sequences. In a de Bruijn graph, all reads are broken into k-mers (all subsequences of length k within the reads) and a path between the k-mers is calculated. In assembly according to this method, the reads are represented as a path through the k-mers. The de Bruijn graph captures overlaps of length k-1 between these k-mers and not between the actual reads. By reducing the entire data set down to k-mer overlaps, the de Bruijn graph reduces the high redundancy in short-read data sets. Assembly of reads using de Bruijn graphs is described in U.S. Pub. 2011/0004413, U.S. Pub. 2011/0015863, and U.S. Pub. 2010/0063742, incorporated by reference in their entirety. Assembly of reads into contigs is further discussed in U.S. Pat. No. 6,223,128, U.S. Pub. 2009/0298064, U.S. Pub. 2010/0069263, and U.S. Pub. 2011/0257889, each of which is incorporated by reference herein in its entirety.

Computer programs for assembling reads are known in the art. Such assembly programs can run on a general-purpose computer, on a cluster or network of computers, or on a specialized computing device dedicated to sequence analysis.

Assembly can be implemented, for example, by the program ‘The Short Sequence Assembly by k-mer search and 3′ read Extension’ (SSAKE), from Canada's Michael Smith Genome Sciences Centre (Vancouver, B.C., CA) (see, e.g., Warren, R., et al., Bioinformatics, 23:500-501 (2007)). SSAKE cycles through a table of reads and searches a prefix tree for the longest possible overlap between any two sequences. SSAKE clusters reads into contigs.

Another read assembly program is Forge Genome Assembler, written by Darren Platt and Dirk Evers and available through the SourceForge web site maintained by Geeknet (Fairfax, Va.) (see, e.g., DiGuistini, S., et al., Genome Biology, 10:R94 (2009)). Forge distributes its computational and memory consumption to multiple nodes, if available, and has therefore the potential to assemble large sets of reads. Forge was written in C++ using the parallel MPI library. Forge can handle mixtures of reads, e.g., Sanger, 454, and Illumina reads.

Other read assembly programs include Clustal Omega, (Sievers F., et al., Mol Syst Biol 7 (2011)), ClustalW, or ClustalX (Larkin M. A., et al., Bioinformatics, 23, 2947-2948 (2007)) available from University College Dublin (Dublin, Ireland); Velvet, available through the web site of the European Bioinformatics Institute (Hinxton, UK) (Zerbino D. R. et al., Genome Research 18(5):821-829 (2008)); programs from the package SOAP, available through the website of Beijing Genomics Institute (Beijing, CN) or BGI Americas Corporation (Cambridge, Mass.); ABySS, from Canada's Michael Smith Genome Sciences Centre (Vancouver, B.C., CA) (Simpson, J. T., et al., Genome Res., 19(6):1117-23 (2009)); and Roche's GS De Novo Assembler, known as gsAssembler or “Newbler” (NEW assemBLER), which is designed to assemble reads from the Roche 454 sequencer (described, e.g., in Kumar, S. et al., Genomics 11:571 (2010) and Margulies, et al., Nature 437:376-380 (2005)). Newbler accepts 454 Flx Standard reads and 454 Titanium reads as well as single and paired-end reads and optionally Sanger reads. Newbler is run on Linux, in either 32 bit or 64 bit versions. Newbler can be accessed via a command-line or a Java-based GUI interface. Other read assembly programs include Cortex; RTG Investigator; iAssembler; TgiCL Assembler; Maq; MIRA3; PGA4genomics; and Phrap.

In aspects of the invention wherein sequence reads are assembled into contigs, as disclosed above, the contig may be aligned to either the primary or a secondary path of the graph genome. Alignment, as used herein, generally involves placing one sequence along another sequence, iteratively introducing gaps along each sequence, scoring how well the two sequences match, and preferably repeating for various positions along the reference. Alignment according to some embodiments of the invention includes pairwise alignment. In some embodiments, pairwise alignment proceeds according to dot-matrix methods, dynamic programming methods, or word methods. Dynamic programming methods may implement the Smith-Waterman (SW) algorithm or the Needleman-Wunsch (NW) algorithm as described, for example, in U.S. Pat. No. 5,701,256 and U.S. Pub. 2009/0119313, both herein incorporated by reference in their entirety.

In certain embodiments, the invention provides methods of alignment which avoid an exhaustive pairwise alignment by making a suffix tree (sometime known as a suffix trie). Given a reference genome T, a suffix tree for T is a tree comprising all suffices of T such that each edge is uniquely labeled with a character, and the concatenation of the edge labels on a path from the root to a leaf corresponds to a unique suffix of T. A Burrows-Wheeler transform (BWT) can be used to index reference T, and the index is used to emulate a suffix tree. One exemplary computer alignment program that implements a BWT is Burrows-Wheeler Aligner (BWA) available from the SourceForge web site maintained by Geeknet (Fairfax, Va.). Alignment by BWA can proceed using the algorithm bwa-short, designed for short queries up to ˜200 base pair with low error rate (<3%) (Li H. and Durbin R. Bioinformatics, 25:1754-60 (2009)). The second algorithm, BWA-SW, is designed for long reads with more errors (Li H. and Durbin R. (2010) Fast and accurate long-read alignment with Burrows-Wheeler Transform. Bioinformatics, Epub.). The BWA-SW component performs heuristic Smith-Waterman-like alignment to find high-scoring local hits. One skilled in the art will recognize that bwa-sw is sometimes referred to as “bwa-long”, “bwa long algorithm”, or similar. Such usage generally refers to BWA-SW.

Other exemplary alignment programs include: Efficient Large-Scale Alignment of Nucleotide Databases (ELAND) or the ELANDv2 component of the Consensus Assessment of Sequence and Variation (CASAVA) software (Illumina, San Diego, Calif.); RTG Investigator; Novoalign; MUMmer; BLAT; SOAP2; and Bowtie.

With each contig aligned to either path of the graph genome, individual reads are aligned to the contigs, and positional and variant information is mapped from the genome to the individual reads through the contig. Once the reads are mapped, the alignments can then be convoluted to the primary path of the graph genome, as shown in Step 3 of FIG. 2.

In another embodiment, methods of the invention include the alignment of raw reads to the primary or secondary path through the graph genome without first assembling the reads into contigs. Once aligned to either the primary or secondary path, the raw read alignments can then be convoluted back down to the primary genomic path.

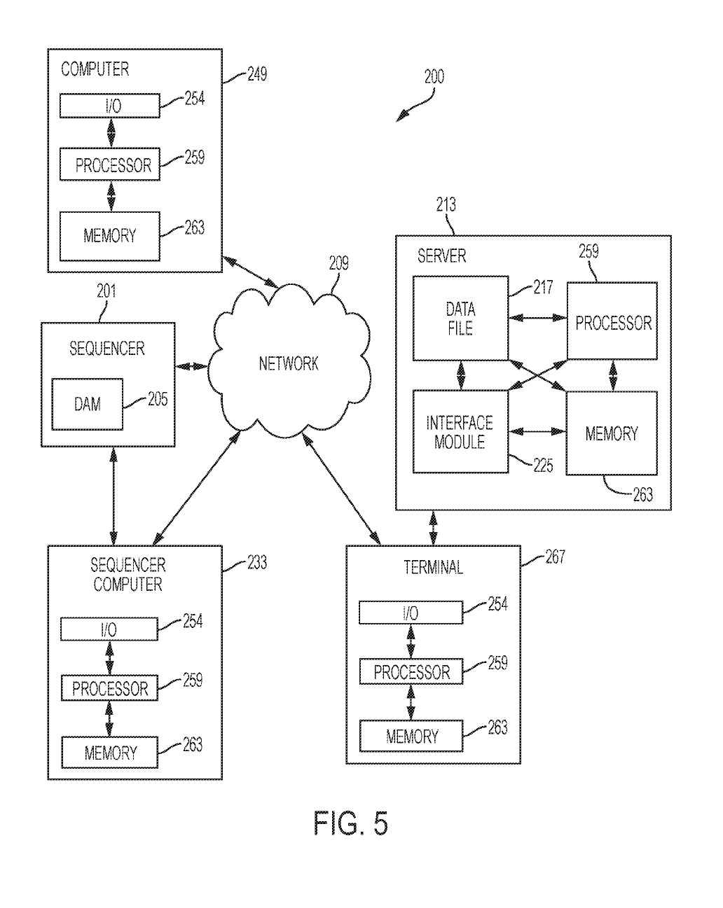

Methods of the invention can be performed using a computer system 200, as shown in FIG. 5. Computer system 200 can optionally include a sequencer 201 with data acquisition module 205 to obtain sequence read data. It should be noted that methods may be performed by a single computer or any combination of elements shown in system 200. Sequencer 201 may optionally include or be operably coupled to its own, e.g., dedicated, sequencer computer 233 (including an input/output mechanism 354, one or more of processor 259 and memory 263). Additionally or alternatively, sequencer 201 may be operably coupled to a server 213 or computer 249 (e.g., laptop, desktop, or tablet) via network 209. Computer 249 includes one or more processor 259 and memory 263 as well as an input/output mechanism 254. Where methods of the invention employ a client/server architecture, any steps of methods of the invention may be performed using server 213, which includes one or more of processor 259 and memory 263, capable of obtaining data, instructions, etc., or providing results via interface module 225 or providing results as a file 217. Server 213 may be engaged over network 209 through computer 249 or terminal 267, or server 213 may be directly connected to terminal 267, including one or more processor 259 and memory 263, as well as input/output mechanism 254.

System 200 or machines therein may include for any of I/O 254 a video display unit (e.g., LED, LCD, or cathode ray tube (CRT)), an alphanumeric input device (e.g., a keyboard), a cursor control device (e.g., a mouse or trackpad), a disk drive unit, a signal generation device (e.g., a speaker), a touchscreen, an accelerometer, a microphone, a cellular radio frequency antenna, network interface device (e.g., a network interface card (NIC), Wi-Fi card, or cellular modem), or combination thereof. Processor 259 can include one or more microchips such as a Intel or AMD processor. Memory 263 can include a non-transitory machine-readable medium on which is stored one or more sets of instructions (e.g., software) embodying any one or more of the methodologies or functions described herein. While the machine-readable medium can in an exemplary embodiment be a single medium, the term “machine-readable medium” should be taken to include a single medium or multiple media (e.g., a centralized or distributed database, and/or associated caches and servers) and may include, without limit, solid-state memories (e.g., subscriber identity module (SIM) card, secure digital card (SD card), micro SD card, or solid-state drive (SSD)), optical and magnetic media, and any other tangible storage media. The software may also reside, completely or at least partially, within the main memory and/or within the processor during execution thereof by the computer system, the main memory and the processor also constituting machine-readable media. The software may further be transmitted or received over a network via the network interface device.

As depicted in FIG. 5, computer system 200 is operable to receive a plurality of sequence reads and introduce a simulated mutation into at least one of the reads. Any one or combination of the computers in system 200 may be programmed to modify sequence read files. A program may be written in any suitable language including compiled or non-complied languages. Suitable programming languages include C, C++, Perl, Python, Ruby, Groovy, Java, other suitable programming languages known in the art, or a combination thereof. Computer system 200 is also operable to process the sequence reads obtained from the sequencer using any known assembly method, including the graph-based alignment method described above and shown in FIG. 2.

A computer program can obtain data describing the mutation(s) to be simulated from any suitable source. In some embodiments, data for simulated mutations is obtained from a genetic database or file of genetic data. In certain embodiments, data is obtained by analyzing sequence reads from prior analyses. Data describing the mutation(s) to be simulated may be obtained by human input (e.g., from reviewing medical literature) or by automated processes (e.g., such as parsing a file or a web source for certain types of information using, for example, pattern matching, which may be implemented through the use of regular expressions).

Systems and methods of the invention may be used to evaluate the validity of (e.g., validate) an analytical method, system, or combination thereof used in genetic testing. Methods of the invention include inserting simulated mutations into a limited number of sequence reads during a genetic analysis to provide a control. Other applications for systems and methods of the invention include testing the detectability of a mutation. For example, where a mutation is known or suspected, whether it is abundant, rare, very rare, or of unknown prevalence, the invention provides the ability to evaluate whether that mutation will be detected by known systems and methods. In certain embodiments, the invention provides the ability to test a proposed genetic analysis method, such as a new molecular assay, a sequencing instrument in development, or to test an assembly or genotyping algorithm by comparison to another algorithm.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLE

NGS was performed on a set of 370 samples to provide sequencing data, including over 2,000 variants. Using the simulation method (GATAsim) described above, variants obtained from a list were introduced into actual sequencing reads at 25% allele fraction to simulate biased capture. The reads were run through a full analysis pipeline, which allowed for end-to-end simulation in the context of actual sequencing/capture error modes. Using this simulation method, 2,171 pathogenic variants across 302 genes were identified.

Using the graph-based alignment method (GATAgraph) described above, variants with less than 100% detection sensitivity were added as separate paths through the genome. This allowed for the detection of 1,955 out of the 2,171 variants with 100% sensitivity. As a comparison, variants were also detected using the Genotyping by Assembly-Templated Alignment (GATA) method. See Umbarger (2013).

Genetic analysis by GATA-based methods, as described above, includes obtaining sequence reads and assembling the reads into a contig, which is then aligned to a reference. Differences are identified by comparison. The raw reads are aligned to the contigs and positional and variant information is mapped to the reads from the reference via the contig, allowing genotyping to produce an observed genotyping. The superiority of GATA-based methods in detecting variants over the more conventional genotyping-by-alignment only methods was described in U.S. Patent Publication No. 2014/0129201 and Umbarger et al., (2013) Next-generation carrier screening, Genetics in Medicine, 16(2), 132-140., both of which are incorporated herein in their entirety.

The table below illustrates the number of variants detected using the two methods—GATA and GATAgraph.

TABLE 1

| Alignment Method | GATA | GATAgraph |
|---|---|---|
| # of Simulated Variants | 2,172 | 2,172 |
| # of Variants Detected with 100% Sensitivity | 1,955 | 2,156 |

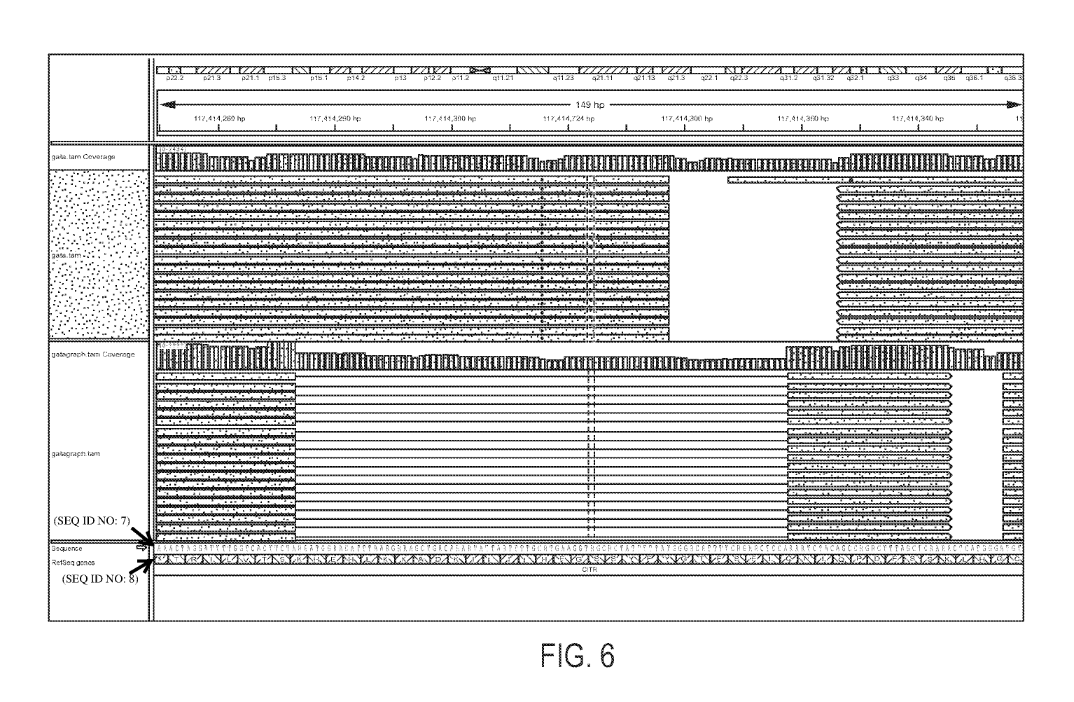

Some of the variants detected using the GATAgraph method that were not detected using GATA include the following: CFTR:c.1817_1900de184; PCCB:c.1278_1291del14ins12; and GBE1:c.2053-3358_2053-3350del9ins19. FIG. 6 illustrates the detection of the CFTR:c.1817_1900de184 variant.

In view of the results provided above, one can see that the graph-based alignment method of this invention can be used to improve the sensitivity of NGS-based assays to detect complex variants. Furthermore, by combining the graph-based alignment method with the simulation method disclosed, identification and detection of complex variants in clinical sequencing can be improved.

What is claimed is:

1. A method for improving variant detection, the method comprising:
generating an in silico genome map comprising:
a primary path; and
a secondary path comprising variants that were missed or incorrectly called in a sequence assembly simulation;
aligning the secondary path to the primary path;
convoluting the second path to the primary path; and
identifying the variants based upon reads that align with the secondary path but not the primary path.

2. The method of claim 1, wherein the aligning step comprises assembling the plurality of sequence reads to produce a contig.

3. The method of claim 2, wherein the aligning step further comprises aligning the contig to either the primary path or the secondary path of the graph genome.

4. The method of claim 3, wherein the aligning step further comprises aligning one or more of the plurality of sequence reads to the contig.

5. The method of claim 1, wherein the computer is operable to report the one or more variants from the plurality of sequence reads.

6. A method for improving variant detection of a genetic test, the method comprising:
generating an in silico genome map:
a primary path; and
a secondary path comprising variants that were missed or incorrectly called in a sequence assembly simulation;
analyzing, using the computer, a plurality of sequence reads generated by sequencing nucleic acid from a sample to determine whether the test is able to identify the variants based on the alignment of the reads to the in silico genome map.

7. The method of claim 6, wherein the analyzing step comprises aligning, using the computer, a plurality of sequence reads to the genome map in a manner that allows the plurality of sequence reads to align to either the primary or secondary paths.

8. The method of claim 7, wherein the analyzing step further comprises convoluting, using the computer, sequence reads that align to the secondary path or the primary path.

9. The method of claim 8, wherein analyzing step further comprises identifying, using the computer, one or more variants from the plurality of sequence reads.
